(19) United States
(12) Patent Application Publication
(10) Pub. No.: US 2026/0209833 A1
(43) Pub. Date: Jul. 23, 2026

(54) METHODS, COMPOSITIONS, AND KITS FOR DETECTING SPATIAL CHROMOSOMAL INTERACTIONS

(71) Applicant: 10x Genomics, Inc., Pleasanton, CA (US)

(72) Inventor: Layla Katiraee, Castro Valley, CA (US)

(21) Appl. No.: 19/570,880

(22) Filed: Mar. 18, 2026

Related U.S. Application Data
(63) Continuation of application No. PCT/US2024/048084, filed on Sep. 24, 2024.
(60) Provisional application No. 63/585,346, filed on Sep. 26, 2023.

(51) Int. Cl. C12Q 1/6837; C12Q 1/34; C12Q 1/6806; C12Q 1/6869; C40B 70/00

(52) U.S. Cl. CPC C12Q 1/6837; C12Q 1/34; C12Q 1/6806; C12Q 1/6869; C40B 70/00; C12Y 301/11; C12Y 301/21001; C12Y 301/31001

(57) ABSTRACT

The present disclosure features methods, compositions, and kits for spatially determining the location of chromosomal conformation interactions in a biological sample.

### CROSS-REFERENCE TO RELATED APPLICATIONS

Pursuant to 35 U.S.C. § 119(e), this application is a continuation of International Application PCT/US2024/048084, with an international filing date of Sep. 24, 2024, which claims the benefit of U.S. Provisional Application No. 63/585,346 filed Sep. 26, 2023, which is herein incorporated by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

Chromosomal conformation capture techniques (e.g., “3C”, “4C”, “5C”, “Hi-C”, etc.) are molecular biology tools used to analyze the organization of chromatin within a cell. These techniques enable quantification of chromosomal conformation interactions between genomic loci that are nearby in three-dimensional space, but can be separated by many nucleotides in the linear genome. Numerous reasons account for such interactions including, for example, promoter-enhancer interactions, chromatin loops, topologically associating domains (TADs), etc. While these chromosome conformation capture techniques have proven useful for understanding epigenetic control, transcriptional regulation, and the large-scale organization of the genome, there remains a need to spatially correlate these chromosomal conformation interactions to a location within a biological sample (e.g., a tissue section).

### SUMMARY

The present disclosure features methods, compositions, and kits for the spatial detection (e.g., a spatial location within a biological sample) of chromosomal conformation interactions. While several chromosomal conformation interaction techniques have been developed, these techniques are unable to identify a spatial location associated with these interactions within a biological sample (e.g., a tissue section). Methods are still needed to understand the spatial location of epigenetic control, transcriptional regulation, and genome organization within a biological sample. Existing chromosomal conformation interaction methods can be adapted such that these interactions can be correlated back to a spatial location within a biological sample. The methods disclosed herein can also be useful to compare diseased biological samples to healthy tissue where further insight can be gleaned from differences in transcriptional regulation, for example. Therefore, the present disclosure features methods, compositions, and kits to spatially identify such chromosomal conformation interactions.

Thus, provided herein are methods for determining a spatial location of chromosomal conformation interactions in a biological sample, the method including: (a) providing an array including a plurality of capture probes, where a capture probe of the plurality of capture probes includes: (i) a spatial barcode and (ii) a capture domain; (b) crosslinking accessible DNA in the biological sample; (c) fragmenting the crosslinked accessible DNA, thereby generating crosslinked fragmented DNA; (d) circularizing the crosslinked fragmented DNA, thereby generating crosslinked circularized DNA; (e) digesting the crosslinked circularized DNA, thereby generating crosslinked digested DNA; (f) incorporating a capture sequence onto an end of the crosslinked digested DNA; (g) hybridizing the capture sequence of the crosslinked digested DNA to the capture domain of the capture probe on the array; and (h) determining (i) the sequence of the spatial barcode, or a complement thereof, and (ii) all or a portion of the sequence of the crosslinked digested DNA, or a complement thereof, and using the determined sequences of (i) and (ii) to determine the spatial location of chromosomal conformation interactions in the biological sample.

In some embodiments, the biological sample is disposed on the array. In some embodiments, the biological sample is disposed on a substrate. In some embodiments, the method includes aligning the substrate including the biological sample with the array, such that at least a portion of the biological sample is aligned with at least a portion of the array.

In some embodiments, the digesting the crosslinked circularized DNA includes treating the biological sample with Exonuclease VIII.

In some embodiments, the circularizing the crosslinked fragmented DNA includes ligating together ends of the crosslinked fragmented DNA.

In some embodiments, the circularizing the crosslinked fragmented DNA includes a gap-fill reaction with one or more nucleotides.

In some embodiments, at least one of the one or more nucleotides includes a biotin moiety. In some embodiments, the method includes an enrichment step where the biotin moiety interacts with a streptavidin moiety.

In some embodiments, the circularizing the crosslinked fragmented DNA includes use of a ligase.

In some embodiments, the incorporating the capture sequence onto the end of the crosslinked digested DNA includes ligating a poly(A) oligonucleotide onto the end of the crosslinked digested DNA. In some embodiments, the incorporating the capture sequence onto the end of the crosslinked digested DNA includes the use of a terminal transferase and a plurality of dATPs.

In some embodiments, the crosslinking the accessible DNA in the biological sample includes use of formaldehyde.

In some embodiments, the fragmenting the crosslinked accessible DNA includes use of a restriction enzyme, a DNase, and/or a micrococcal nuclease (MNase).

In some embodiments, the digesting in (e) includes use of a restriction enzyme or a DNase. In some embodiments, the one or more restriction enzymes is selected from the group including: AlwI, Alw26, BamHI, BbsI, BbvI, BceAI, BmrI, Bsal, Bst71, BsmAI, BsmBI, BsmFI, BspMI, EarI, FauI, FokI, HgaI, PleI, SapI, SfaNI, or Sth132.

In some embodiments, the capture probe includes one or more functional domains, a unique molecular identifier, a cleavage domain, or a combination thereof. In some embodiments, the one or more functional domains includes a primer binding site or a sequencing specific site.

In some embodiments, the capture domain includes a poly(T) sequence.

In some embodiments, the array includes a plurality of features selected from the group consisting of: a spot, an inkjet spot, a masked spot, a pit, a post, a well, a ridge, a divot, a hydrogel pad, and a bead.

In some embodiments, the determining step includes sequencing. In some embodiments, the sequencing includes high-throughput sequencing. In some embodiments, the determining step includes fluorescent detection.

In some embodiments, the biological sample is a tissue sample. In some embodiments, the tissue sample is a fixed tissue sample. In some embodiments, the tissue sample is a fresh-frozen tissue sample. In some embodiments, the tissue sample is a tissue section. In some embodiments, the tissue section is a fresh-frozen tissue section. In some embodiments, the tissue section is a fixed tissue section.

In some embodiments, the fixed tissue section is a formalin-fixed paraffin-embedded (FFPE) tissue section, a paraformaldehyde-fixed tissue section, an acetone-fixed tissue section, a methanol-fixed tissue section, or an ethanol-fixed tissue section. In some embodiments, the FFPE tissue section is deparaffinized and decrosslinked prior to (b).

In some embodiments, the method includes staining the biological sample. In some embodiments, the staining includes hematoxylin and/or eosin staining. In some embodiments, the staining includes use of a radioisotope, a fluorophore, a chemiluminescent compound, a bioluminescent compound, or a combination thereof.

In some embodiments, the method includes imaging the biological sample.

In some embodiments, the method includes permeabilizing the biological sample. In some embodiments, the permeabilizing includes use of a protease, a surfactant, and/or a detergent. In some embodiments, the protease includes Proteinase K, pepsin, and/or collagenase.

Also provided herein are kits including: (a) a spatial array including a plurality of capture probes, where a capture probe of the plurality of capture probes includes: (i) a spatial barcode and (ii) a capture domain; (b) one or more restriction enzymes, a DNase, and/or a MNase; (c) a ligase; and (d) Exonuclease VIII.

In some embodiments, the kit includes the one or more restriction enzymes. In some embodiments, the one or more restriction enzymes includes AlwI, Alw26, BamHI, BbsI, BbvI, BceAI, BmrI, Bsal, Bst71, BsmAI, BsmBI, BsmFI, BspMI, EarI, FauI, FokI, HgaI, PleI, SapI, SfaNI, or Sth132.

In some embodiments, the kit includes the DNase. In some embodiments, the MNase. In some embodiments, the kit includes a polymerase.

In some embodiments, the kit includes one or more nucleotides. In some embodiments, at least one of the one or more nucleotides includes a biotin moiety.

In some embodiments, the kit includes a plurality of poly(A) oligonucleotides.

In some embodiments, the kit includes a terminal transferase enzyme. In some embodiments, the kit includes a plurality of dATPs.

In some embodiments, the kit includes one or more crosslinking agents. In some embodiments, the one or more crosslinking agents includes formaldehyde.

Also provided herein are compositions including: a) a spatial array including a plurality of capture probes, where a capture probe of the plurality of capture probes includes: (i) a spatial barcode and (ii) a capture domain; b) a crosslinking agent; and c) crosslinked accessible DNA.

Also provided herein are compositions including: a) a spatial array including a plurality of capture probes, where a capture probe of the plurality of capture probes includes: (i) a spatial barcode and (ii) a capture domain; b) one or more restriction enzymes, a DNase, and/or a MNase; and c) crosslinked fragmented DNA.

Also provided herein are compositions including: a) a spatial array including a plurality of capture probes, where a capture probe of the plurality of capture probes includes: (i) a spatial barcode and (ii) a capture domain; b) one or more restriction enzymes and/or a DNase; and c) crosslinked circularized DNA.

In some embodiments, the composition includes the one or more enzymes. In some embodiments, the one or more restriction enzymes include AlwI, Alw26, BamHI, BbsI, BbvI, BceAI, BmrI, Bsal, Bst71, BsmAI, BsmBI, BsmFI, BspMI, EarI, FauI, FokI, HgaI, PleI, SapI, SfaNI, or Sth132.

In some embodiments, the composition includes the MNase. In some embodiments, the composition includes a ligase. In some embodiments, the composition includes a polymerase.

In some embodiments, the composition includes one or more dNTPs. In some embodiments, at least one dNTP of the one or more dNTPs includes a biotin moiety.

In some embodiments, the composition includes an exonuclease. In some embodiments, the exonuclease is Exonuclease VIII.

Also provided herein are compositions including: a) a spatial array including a plurality of capture probes, where a capture probe of the plurality of capture probes includes: (i) a spatial barcode and (ii) a capture domain; b) crosslinked digested DNA; and c) either: (i) a ligase and a poly(A) oligonucleotide, or (ii) a terminal transferase and a plurality of dATPs.

In some embodiments, the composition includes (i): the ligase and the poly(A) oligonucleotide. In some embodiments, the composition includes (ii): the terminal transferase and the plurality of dATPs.

Also provided herein are compositions including: a) a spatial array including a plurality of capture probes, where a capture probe of the plurality of capture probes includes: (i) a spatial barcode and (ii) a capture domain; and b) crosslinked digested DNA hybridized to the capture domain of the capture probe via a capture sequence.

Also provided herein are compositions including: a) a spatial array including a plurality of capture probes, where a capture probe of the plurality of capture probes includes: (i) a spatial barcode and (ii) a capture domain; (b) one or more restriction enzymes, a DNase, and/or a MNase; (c) a ligase; and (d) Exonuclease VII.

In some embodiments, the composition includes the one or more enzymes. In some embodiments, the one or more restriction enzymes include AlwI, Alw26, BamHI, BbsI, BbvI, BceAI, BmrI, Bsal, Bst71, BsmAI, BsmBI, BsmFI, BspMI, EarI, FauI, FokI, HgaI, PleI, SapI, SfaNI, or Sth132.

In some embodiments, the composition includes the DNase. In some embodiments, the composition includes the MNase. In some embodiments, the composition includes a polymerase.

In some embodiments, the composition includes one or more nucleotides. In some embodiments, the one or more nucleotides includes a biotin moiety. In some embodiments, the one or more nucleotides are one or more dNTPs.

In some embodiments, the composition includes a plurality of poly(A) oligonucleotides.

In some embodiments, the composition includes a terminal transferase enzyme. In some embodiments, the composition includes a plurality of dATPs.

In some embodiments, the composition includes one or more crosslinking agents. In some embodiments, the one or more crosslinking agents includes formaldehyde.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to +5%, and more preferably still up to +1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “substantially complementary” used herein means that a first sequence is at least 50%, 55%, 60%, 65%, 70%, 75%, 80%, 85%, 90%, 95%, 97%, 98%, or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows an exemplary workflow for performing templated capture and producing a ligation product.

FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

FIG. 12 shows an exemplary workflow for detecting chromosomal conformation interactions within a biological sample on a spatial array.

### DETAILED DESCRIPTION

### A. Spatial Analysis Methods

Spatial analysis methodologies described herein can provide a vast amount of analyte expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding or hybridizing to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Patent Application Publication Nos. WO 2018/091676, WO 2020/176788, WO 2017/144338, and WO 2016/057552; Non-patent literature references Rodriques et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev F, dated January 2022); and/or the Visium Spatial Gene Expression Reagent Kits—Tissue Optimization User Guide (e.g., Rev E, dated February 2022), both of which are available at the 10x Genomics Support Documentation website, and can be used herein in any combination, and each of which is incorporated herein by reference in its entirety. Further non-limiting aspects of spatial analysis methodologies and compositions are described herein.

Some general terminology that may be used in this disclosure can be found in Section (I)(b) of PCT Patent Application Publication No. WO 2020/176788 and/or U.S. Patent Application Publication No. 2020/0277663, which is herein incorporated by reference. Typically, a “barcode” is a label, or identifier, which conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Patent Application Publication No. WO 2020/176788 and/or U.S. Patent Application Publication No. 2020/0277663, which is herein incorporated by reference. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these tissue cores into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section. In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix, e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example methanol. In some embodiments, instead of methanol, acetone, or an acetone-methanol mixture can be used. In some embodiments, the fixation is performed after sectioning. In some instances, when the biological sample is fixed using a fixative including an alcohol (e.g., methanol or acetone-methanol mixture), the biological sample is not decrosslinked afterward. In some preferred embodiments, the biological sample is fixed using a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample, e.g., using acetone and/or alcohol (e.g., methanol, acetone-methanol) is performed while the sample is mounted on a substrate (e.g., glass slide, such as a positively charged glass slide).

In some embodiments, the biological sample, e.g., the tissue sample, is fixed, e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing e.g., by formalin or PFA, the biological sample is dehydrated via sucrose gradient. In some instances, the fixed biological sample is treated with a sucrose gradient and then embedded in a matrix, e.g., OCT compound. In some instances, the fixed biological sample is not treated with a sucrose gradient, but rather is embedded in a matrix, e.g., OCT compound, after fixation. In some embodiments, when a fixed frozen tissue sample is treated with a sucrose gradient, the sample can be rehydrated with an ethanol gradient. In some embodiments, the PFA or formalin fixed biological sample, which can be optionally dehydrated via sucrose gradient and/or embedded in OCT compound, is then frozen, e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated using an ethanol gradient.

In some instances, the biological sample (e.g., a fixed frozen tissue sample) is treated with a citrate buffer. Citrate buffer can be used to decrosslink antigens and fixation medium in the biological sample for antigen retrieval. Thus, any suitable decrosslinking agent can be used in addition to or alternatively to citrate buffer. In some embodiments, for example, the biological sample (e.g., a fixed frozen tissue sample) is decrosslinked using TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, the sample is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, the sample can be rehydrated using an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, acid and a soluble organic compound that preserves morphology of biomolecules. PAXgene provides a two-reagent fixative system in which tissue is firstly fixed in a solution containing methanol and acetic acid then stabilized in a solution containing ethanol. See, Ergin B. et al., J Proteome Res. 2010 Oct. 1; 9 (10):5188-96; Kap M. et al., PLOS One.; 6 (11):e27704 (2011); and Mathieson W. et al., Am J Clin Pathol.; 146 (1):25-40 (2016), each of which is hereby incorporated by reference in its entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample is fixed, for example in methanol, acetone, acetone-methanol, PFA, PAXgene or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RNA-templated ligation (RTL) methods disclosed herein. A limitation of direct RNA capture for fixed samples is that the RNA integrity of fixed (e.g., FFPE) samples can be lower than a fresh sample. As such, capturing RNA directly from fixed samples, e.g., by capture of a common sequence, such as a poly(A) tail of an mRNA molecule, can be more difficult. By utilizing RTL probes that hybridize to RNA target sequences in the transcriptome, RNA analytes can be captured without requiring that both a poly(A) tail and target sequences remain intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Non-limiting examples of stains include histological stains (e.g., hematoxylin and/or eosin) and immunological stains (e.g., fluorescent stains). The biological sample can be stained using Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. In some instances, PAS staining is performed after formalin or acetone fixation. In some embodiments, a biological sample (e.g., a fixed and/or stained biological sample) can be imaged. Biological samples are also described in Section (I)(d) of PCT Patent Application Publication No. WO 2020/176788 and/or U.S. Patent Application Publication No. 2020/0277663, which is herein incorporated by reference.

The following embodiments can be used with any of the methods described herein. In some embodiments, the biological sample is imaged. In some embodiments, the biological sample is visualized or imaged using bright field microscopy. In some embodiments, the biological sample is visualized or imaged using fluorescence microscopy. The biological sample can be visualized or imaged using additional methods of visualization and imaging known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes as disclosed herein.

In some embodiments, the method includes staining the biological sample. In some embodiments, the staining includes the use of hematoxylin and/or eosin. In some embodiments, a biological sample can be stained using any number of biological stains, including but not limited to, acridine orange, Bismarck brown, carmine, coomassie blue, cresyl violet, DAPI (4′,6-diamidino-2-phenylindole), eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label, such as a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Patent Application Publication No. WO 2020/176788 and/or U.S. Patent Application Publication No. 2020/0277663, which is herein incorporated by reference. Briefly, in any of the methods described herein, the method includes a step of permeabilizing the biological sample. For example, the biological sample can be permeabilized to facilitate transfer of the extension products to the capture probes on the array. In some embodiments, the permeabilizing includes the use of an organic solvent (e.g., acetone, ethanol, or methanol), a detergent (e.g., saponin, Triton X-100™, Tween-20™, or sodium dodecyl sulfate (SDS)), an enzyme (e.g., an endopeptidase, an exopeptidase, or a protease), or a combination thereof. In some embodiments, the permeabilizing includes the use of an endopeptidase, a protease, SDS, polyethylene glycol tert-octylphenyl ether, polysorbate 80, and polysorbate 20, N-lauroylsarcosine sodium salt solution, saponin, Triton X-100™, Tween-20™, or a combination thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, which is incorporated herein by reference.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Patent Application Publication No. WO 2020/176788 and/or U.S. Patent Application Publication No. 2020/0277663, which is herein incorporated by reference. Generation of capture probes can be achieved by any appropriate method, including those described in Section (II)(d)(ii) of PCT Patent Application Publication No. WO 2020/176788 and/or U.S. Patent Application Publication No. 2020/0277663, which is herein incorporated by reference.

In some instances, a capture probe and a nucleic acid analyte interaction (or any other nucleic acid to nucleic acid interaction) occurs because the sequences of the two nucleic acids are substantially complementary to one another. By “substantial,” “substantially” and the like, two nucleic acid sequences can be complementary when at least 60% of the nucleotide residues of one nucleic acid sequence are complementary to nucleotide residues of the other nucleic acid sequence. The complementary residues within a particular complementary nucleic acid sequence need not always be contiguous with each other, but can be interrupted by one or more non-complementary residues within the complementary nucleic acid sequence. In some embodiments, at least 60%, but less than 100%, of the residues of one of the two complementary nucleic acid sequences are complementary to residues of the other nucleic acid sequence. In some embodiments, at least 70%, 80%, 90%, 95%, or 99% of the residues of one nucleic acid sequence are complementary to residues of the other nucleic acid sequence. Sequences are said to be “substantially complementary” when at least 60% (e.g., at least 70%, at least 80%, or at least 90%) of the residues of one nucleic acid sequence are complementary to residues in the other nucleic acid sequence.

In some embodiments, the biological sample is mounted on a first substrate and the substrate comprising the array of capture probes is a second substrate. In this configuration, one or more analytes or analyte derivatives (e.g., intermediate agents, e.g., ligation products) can then be released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described e.g., in U.S. Patent Application Publication No. 2021/0189475 and PCT Patent Application Publication Nos. WO 2021/252747 A1, WO 2022/061152 A2, and WO 2022/140028 A1, each of which is herein incorporated by reference.

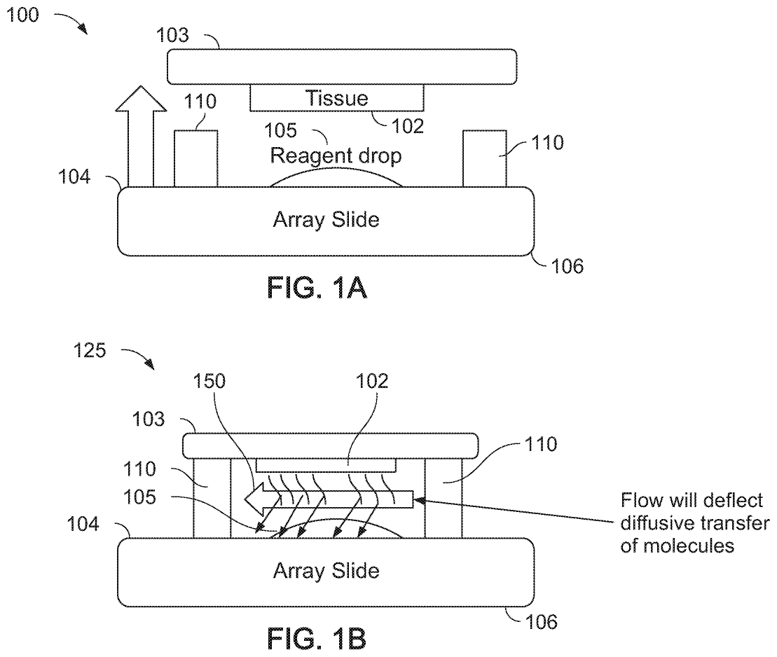

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A, a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents, e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments, wherein the biological sample 102 has been pre-permeabilized, the reagent medium is not a permeabilization solution. In some embodiments, analytes (e.g., mRNA transcripts) and/or analyte derivatives (e.g., intermediate agents, e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent, e.g., ligation product) from the biological sample is performed actively (e.g., electrophoretic, by applying an electric field to promote migration). Exemplary methods of electrophoretic migration are described in PCT Patent Publication No. WO 2020/176788, and U.S. Patent Application Publication No. 2021/0189475, each of which is herein incorporated by reference.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 microns.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate (e.g., slide 103), and the second substrate (e.g., slide 104) may reduce or prevent flow from undesirable movement (e.g., convective movement) of transcripts and/or molecules during the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., U.S. Patent Application Publication No. 2021/0189475, and PCT Patent Publication No. WO 2022/061152 A2, each of which is incorporated by reference in its entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

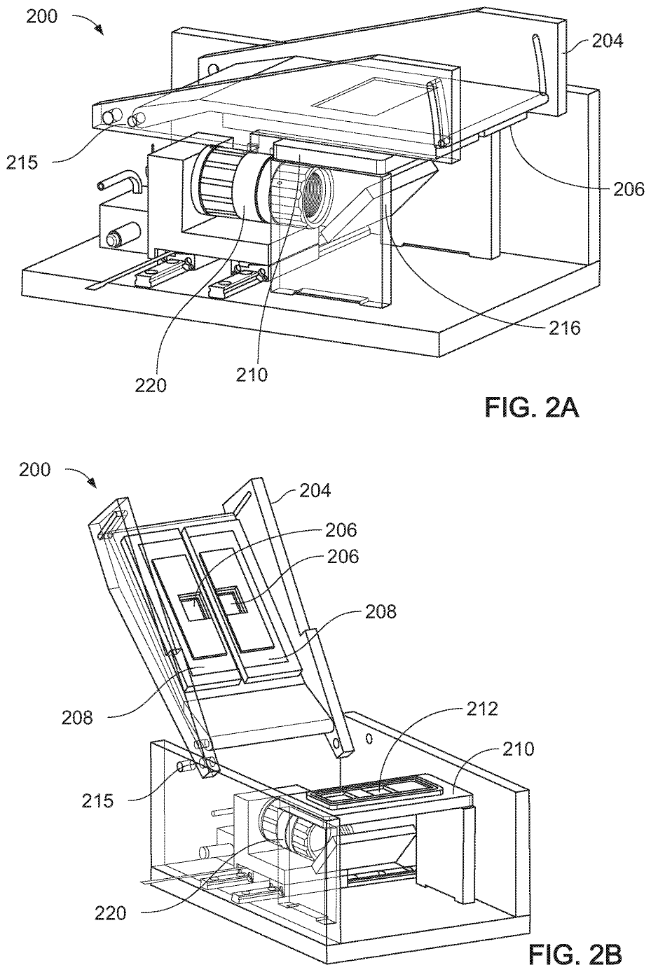

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200, such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents, e.g., ligation products) may be captured by the capture probes of the array and may be processed for spatial analysis.

In some embodiments, during permeabilization, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

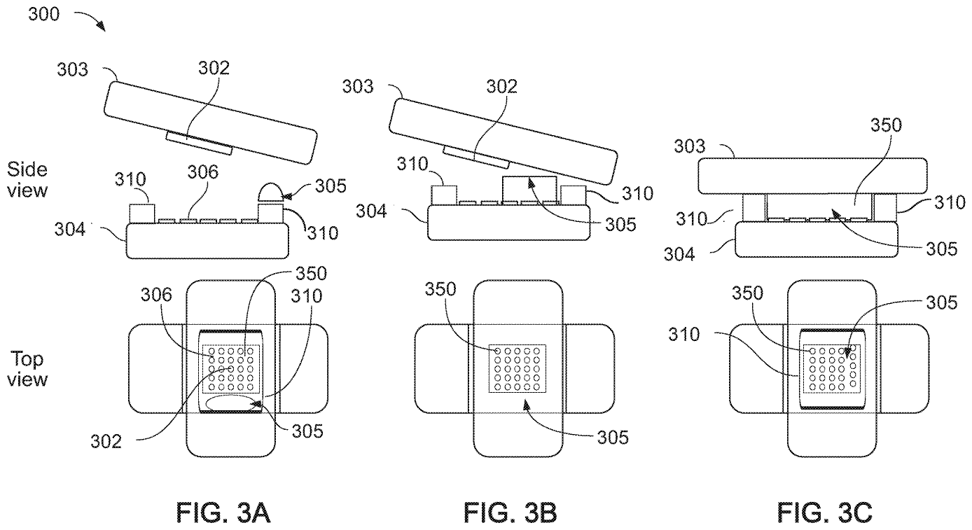

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., the slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right-hand side of side view, it should be understood that such depiction is not meant to be limiting as to the location of the reagent medium on the spacer.

FIG. 3B shows that as the first substrate lowers, and/or as the second substrate rises, the dropped side of the first substrate (e.g., a side of the slide 303 angled toward the slide 304) may contact the reagent medium 305. The dropped side of the slide 303 may urge the reagent medium 305 toward the opposite direction (e.g., towards an opposite side of the spacer 310, towards an opposite side of the slide 303 relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step. In some aspects, bubble formation between the substrates may be reduced or eliminated using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

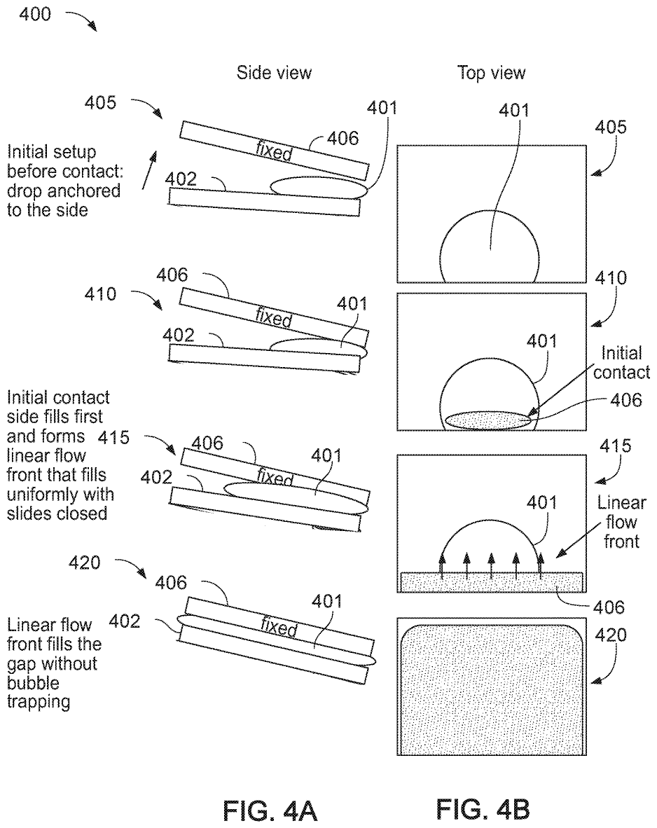

FIG. 4A is a side view of the angled closure workflow 400 in accordance with some exemplary implementations. FIG. 4B is a top view of the angled closure workflow 400 in accordance with some exemplary implementations. As shown at step 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills the gap between the two substrates 406 and 402 uniformly with the slides closed.

At step 415, the substrate 406 is further lowered toward the substrate 402 (or the substrate 402 is raised up toward the substrate 406) and the dropped side of the substrate 406 may contact and may urge the reagent medium toward the side opposite the dropped side, thereby creating a linear or low curvature flow front that may prevent or reduce bubble trapping between the substrates.

At step 420, the reagent medium 401 fills the gap between the substrate 406 and the substrate 402. The linear flow front of the liquid reagent may be formed by squeezing the reagent medium 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, or methanol), cross-linking agents (e.g., paraformaldehyde), detergents (e.g., saponin, Triton X-100™, Tween-20™, or sodium dodecyl sulfate (SDS)), and enzymes (e.g., trypsin or other proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, elastase, and proteinase K. In some embodiments, the reagent medium comprises a nuclease. In some embodiments, the nuclease comprises an RNase. In some embodiments, the RNase includes RNase A, RNase C, RNase H, or RNase I. In some embodiments, the reagent medium comprises one or more of sodium dodecyl sulfate (SDS) or a sodium salt thereof, proteinase K, pepsin, N-lauroylsarcosine, or RNase.

In some embodiments, the reagent medium comprises polyethylene glycol (PEG). In some embodiments, the PEG molecular weight is from about 2K to about 16K. In some embodiments, the PEG molecular weight is about 2K, about 3K, about 4K, about 5K, about 6K, about 7K, about 8K, about 9K, about 10K, about 11K, about 12K, about 13K, about 14K, about 15K, or about 16K. In some embodiments, the PEG is present at a concentration from about 2% to about 25%, from about 4% to about 23%, from about 6% to about 21%, or from about 8% to about 20% (v/v).

In certain embodiments, a dried permeabilization reagent is applied or formed as a layer on the first substrate, the second substrate, or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location in a biological sample. One method is to promote analytes or analyte proxies (e.g., intermediate agents) out of a cell and towards a spatially-barcoded array (e.g., including spatially-barcoded capture probes). Another method is to release or cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Patent Publication No. WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to a terminus (e.g., a 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using a reverse transcriptase. In some embodiments, the capture probe is extended using one or more DNA polymerases. In some embodiments, the extended capture probes include the sequence of the capture domain and the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Patent Application Publication No. WO 2020/176788 and/or U.S. Patent Application Publication No. 2020/0277663, which is herein incorporated by reference. Analysis of captured analytes (and/or intermediate agents or portions thereof), for example, including sample removal, extension of capture probes using the captured analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Patent Application Publication No. WO 2020/176788 and/or U.S. Patent Application Publication No. 2020/0277663, which is herein incorporated by reference. Some quality control measures are described in Section (II)(h) of PCT Patent Application Publication No. WO 2020/176788 and/or U.S. Patent Application Publication No. 2020/0277663, which is herein incorporated by reference.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660, each of which is herein incorporated by reference.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up-regulated and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of PCT Patent Application Publication No. WO 2020/176788 and/or U.S. Patent Application Publication No. 2020/0277663, which is herein incorporated by reference. Exemplary features and geometric attributes of an array can be found in Sections (II)(d)(i), (II)(d)(iii), and (II)(d)(iv) of PCT Patent Application Publication No. WO 2020/176788 and/or U.S. Patent Application Publication No. 2020/0277663, which is herein incorporated by reference.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of PCT Patent Application Publication No. WO 2020/176788 and/or U.S. Patent Application Publication No. 2020/0277663, which is herein incorporated by reference.

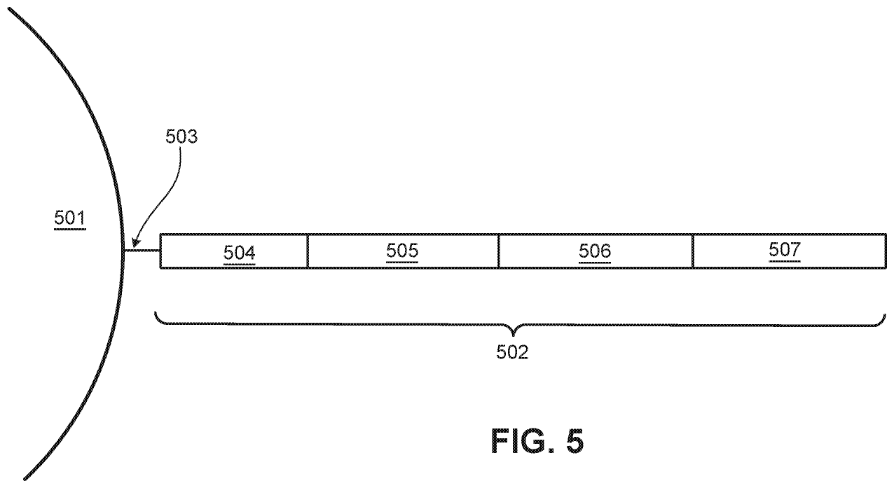

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or a combination thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

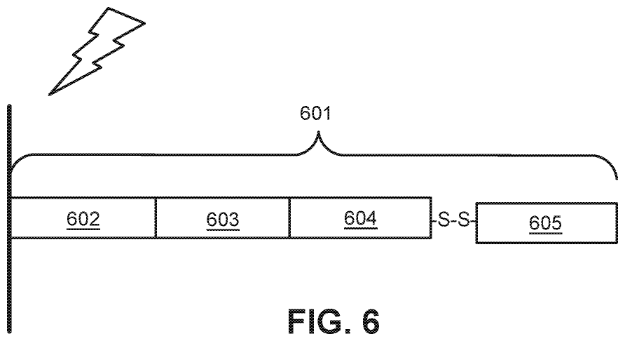

FIG. 6 is a schematic illustrating a cleavable capture probe, wherein the cleaved capture probe can enter into a non-permeabilized cell and bind to analytes within the sample. The capture probe 601 can contain a cleavage domain 602, a cell penetrating peptide 603, a reporter molecule 604, and a disulfide bond (—S—S—). 605 represents all other parts of a capture probe, for example a spatial barcode and a capture domain.

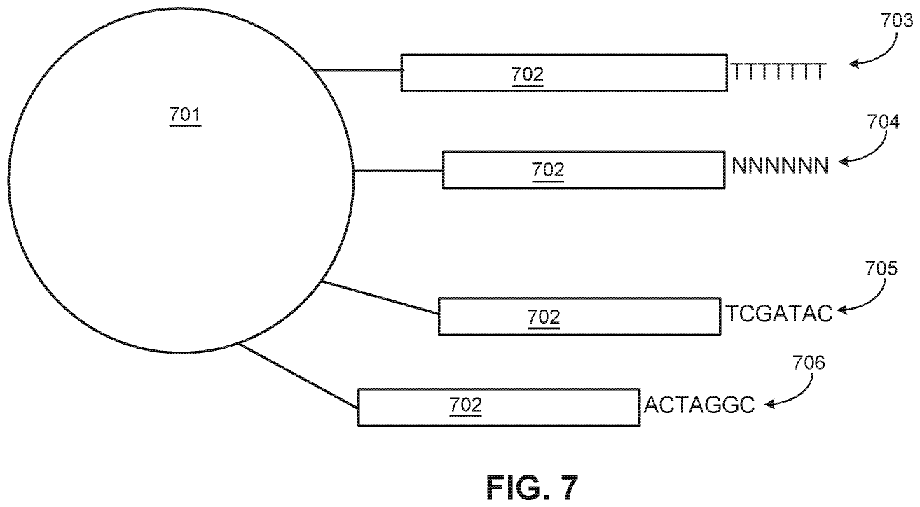

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature can include the spatial barcode 702 in combination with a poly(T) capture domain 703, designed to capture mRNA target analytes. A second type of capture probe associated with the feature can include the spatial barcode 702 in combination with a random N-mer capture domain 704 for gDNA analysis. A third type of capture probe associated with the feature can include the spatial barcode 702 in combination with a capture domain complementary to the analyte capture agent of interest 705. A fourth type of capture probe associated with the feature can include the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and/or metabolites, and gDNA; (b) mRNA, accessible chromatin (e.g., ATAC-seq, DNase-seq, and/or MNase-seq), cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V (D) J sequence of an immune cell receptor (e.g., T-cell receptor). In some embodiments, a perturbation agent can be a small molecule, an antibody, a drug, an aptamer, a miRNA, a physical environmental (e.g., temperature) change, or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used include, but are not limited to, Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequences 504 are common to all of the probes attached to a given feature. In some embodiments, the UMI sequence 506 of a capture probe attached to a given feature is different from the UMI sequence of a different capture probe attached to the given feature.

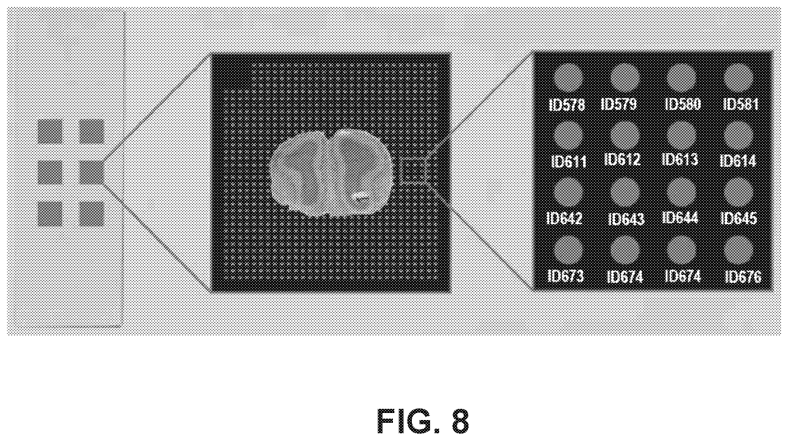

FIG. 8 depicts an exemplary arrangement of barcoded features within an array. From left to right, FIG. 8 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (e.g., labelled as ID578, ID579, ID580, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Patent Application Publication No. WO 2020/176788 and/or U.S. Patent Application Publication No. 2020/0277663, which is herein incorporated by reference.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Patent Application Publication No. WO 2020/176788 and/or U.S. Patent Application Publication No. 2020/0277663, which is herein incorporated by reference.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128, which is herein incorporated by reference. RTL may include hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence or a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using potassium hydroxide (KOH). The released ligation product can then be captured by capture probes (e.g., instead of direct capture of an analyte) on an array, optionally amplified, and sequenced, thus determining the location and optionally the abundance of the analyte in the biological sample.

In some instances, one or both of the oligonucleotides may hybridize to genomic DNA (gDNA) which can lead to false positive sequencing data from ligation events on gDNA (off target) in addition to the desired (on target) ligation events on target nucleic acids (e.g., mRNA). Thus, in some embodiments, the disclosed methods can include contacting the biological sample with a deoxyribonuclease (DNase). The DNase can be an endonuclease or exonuclease. In some embodiments, the DNase digests single-stranded and/or double-stranded DNA. Suitable DNases include, without limitation, a DNase I and a DNase II. Use of a DNase as described can mitigate false positive sequencing data from off target gDNA ligation events.

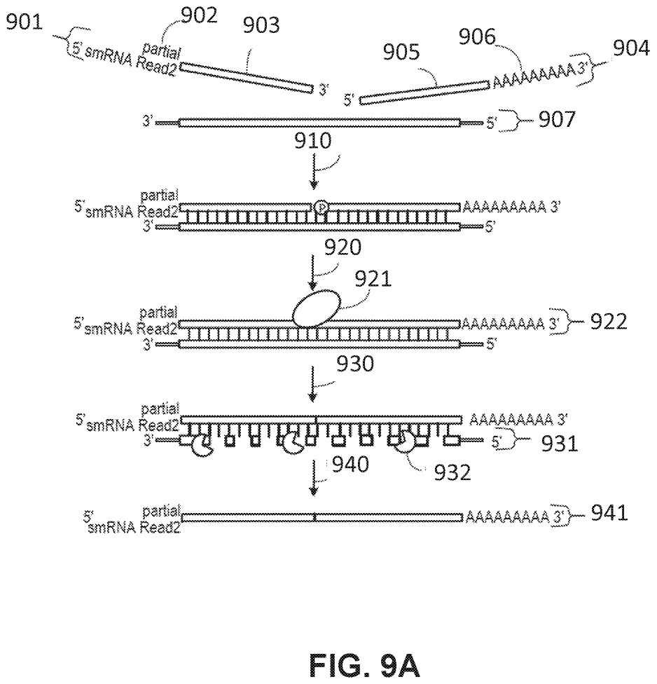

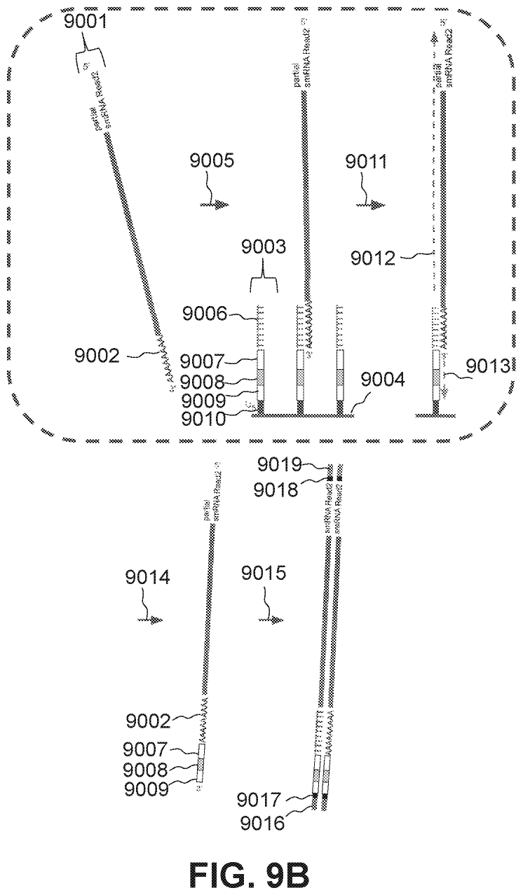

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly(A) sequence) 906, the first probe 901 and the second probe 904 hybridize 910 to an analyte 907. A ligase 921 ligates 920 the first probe 901 to the second probe 904, thereby generating a ligation product 922. The ligation product 922 is then released 930 from the analyte 931 by digesting the analyte 907 using an endoribonuclease 932. The sample is permeabilized 940 and the ligation product 941 is able to hybridize to a capture probe on the substrate. Methods and composition for spatial detection using templated ligation have been described in PCT Patent Application Publication No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product 9001 specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily capture the target analytes (i.e., compared to no permeabilization). In some embodiments, reverse transcription (RT) reagents can be added to permeabilized biological samples. Incubation with the RT reagents can be used to extend the capture probes 9011 to produce spatially-barcoded full-length cDNA 9012 and 9013 from the captured ligation products (e.g., polyadenylated mRNA ligation products). Second strand reagents (e.g., second strand primers, enzymes, etc.) can be added to the biological sample to initiate second strand synthesis.

In some embodiments, methods provided herein include permeabilization of the biological sample such that the capture probe can more easily capture the ligation products (i.e., compared to no permeabilization). In some embodiments, reverse transcription (RT) reagents can be added to permeabilize biological samples. Incubation with the RT reagents can be used to extend the capture probes 9011 to produce spatially-barcoded full-length cDNA 9012 and 9013 from the captured ligation products (e.g., polyadenylated ligation products).

In some embodiments, the extended ligation products can be denatured 9014, released from the capture probe and transferred (e.g., to a clean tube) for amplification, and/or library construction. The spatially-barcoded ligation products can be amplified 9015 via PCR prior to library construction. P5 9016, i5 9017, i7 9018, and P7 9019 can be used as flow cell capture sequences and sample indexes. The amplicons can then be sequenced using paired-end sequencing using TruSeq Read 1 and TruSeq Read 2 as sequencing primer sites, for example.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Patent Application Publication No. WO 2020/176788 and/or Section (II)(b)(viii) U.S. Patent Application Publication No. 2020/0277663, which is herein incorporated by reference.

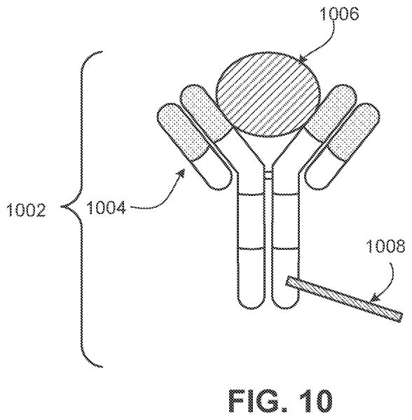

FIG. 10 is a schematic diagram of an exemplary analyte capture agent 1002 comprised of an analyte binding moiety 1004 and an analyte binding moiety barcode domain 1008. The exemplary analyte binding moiety 1004 is a molecule capable of binding to an analyte 1006 and the analyte capture agent 1002 is capable of interacting with a spatially-barcoded capture probe on an array. The analyte binding moiety 1004 can bind to the analyte 1006 with high affinity and/or with high specificity. The analyte capture agent 1002 can include: (i) an analyte binding moiety barcode domain 1008 which serves to identify the analyte binding moiety, and an analyte capture sequence, which can hybridize to at least a portion or an entirety of a capture domain of a capture probe. The analyte binding moiety 1004 can include a polypeptide and/or an aptamer. The analyte binding moiety 1004 can include an antibody or antibody fragment (e.g., an antigen-binding fragment).

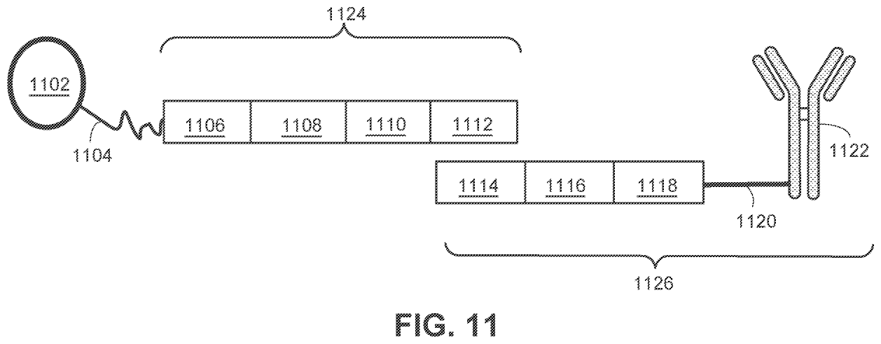

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequences 1106 and a UMI 1110, as described elsewhere herein. The capture probe can be affixed 1104 to a feature such as a bead 1102. The capture probe 1124 can also include a capture domain 1112 that is capable of binding to an analyte capture agent 1126. The analyte-binding moiety barcode domain of the analyte capture agent 1126 can include a functional sequence 1118, analyte binding moiety barcode 1116, and an analyte capture sequence 1114 that is capable of binding (e.g., hybridizing) to the capture domain 1112 of the capture probe 1124. The analyte capture agent 1126 can also include a linker 1120 that allows the analyte binding moiety barcode domain (e.g., including the functional sequence 1118, analyte binding moiety barcode 1116, and analyte capture sequence 1114) to couple to the analyte binding moiety 1122. In some embodiments, the linker 1120 is a cleavable linker. In some embodiments, the cleavable linker is a photo-cleavable linker, a UV-cleavable linker, chemical-cleavable linker, thermal-cleavable linker, or an enzyme cleavable linker. In some instances, the cleavable linker is a disulfide linker. A disulfide linker can be cleaved by use of a reducing agent, such as dithiothreitol (DTT), beta-mercaptoethanol (BME), or tris(2-carboxyethyl)phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that each spatial barcode is uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of PCT Patent Application Publication No. WO 2020/176788 and/or U.S. Patent Application Publication No. 2020/0277663, which is herein incorporated by reference. See, for example, the Exemplary embodiment starting with “In some non-limiting examples of the workflows described herein, the sample can be immersed . . . ” of PCT Patent Application Publication No. WO 2020/176788 and/or U.S. Patent Application Publication No. 2020/0277663, which is herein incorporated by reference. See also, e.g., the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022), each of which is herein incorporated by reference in its entirety.

In some embodiments, spatial analysis can be performed using dedicated hardware and/or software, such as any of the systems described in Sections (II)(e)(ii) and/or (V) of PCT Patent Application Publication No. WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Patent Application Publication No. WO 2020/123320, which is herein incorporated by reference.

Suitable systems for performing spatial analysis can include components such as a chamber (e.g., a flow cell or a sealable, fluid-tight chamber) for containing a biological sample. The biological sample can be mounted, for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable, and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD or CMOS) used to capture images. The systems can also optionally include one or more light sources (e.g., LED-based, diode-based, or lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Patent Application Publication No. WO 2021/102003 and/or U.S. Patent Application Publication No. 2021/0150707, each of which is incorporated herein by reference in its entirety.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-dimensional and/or three-dimensional map of the analyte presence and/or level are described in PCT Patent Application Publication No. WO 2020/053655 and spatial analysis methods are generally described in PCT Patent Application Publication No. WO 2021/102039 and/or U.S. Patent Application Publication No. 2021/0155982, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Patent Application Publication Nos. WO 2020/123320, WO 2021/102005, and/or U.S. Patent Application Publication No. 2021/0158522, each of which is incorporated herein by reference in its entirety. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### B. Methods of Detecting the Spatial Location of Chromosomal Conformation Interactions in a Biological Sample

The present disclosure features methods, compositions, and kits for detecting and determining the spatial location of chromosomal conformation interactions within a biological sample (e.g., a tissue sample). In particular, the methods disclosed herein can be used to identify chromosomal conformation interactions within a biological sample (e.g., within different cell types of a biological sample), compare chromosomal conformation interaction profiles between diseased and healthy tissue, and elucidate epigenetic regulation within a biological sample among other biological processes.

Several chromosomal conformation capture methods have been developed including 3C, 4C, 5C, and Hi-C, as well as other derivative assays. Chromosome conformation capture (“3C”) assays quantify interactions between a single pair of genomic loci. For example, 3C can be used to test a candidate promoter-enhancer interaction and ligated fragments may be detected using PCR with known primers (Dekker, J., et al., Capturing chromosome conformation, Science, 295 (5558), 1306-11 (2002)). 3C assays require prior knowledge of the interacting regions of a given pair of genomic loci. Chromosome conformation capture-on-chip (“4C”), on the other hand, captures interactions between one locus and all other genomic loci. 4C assays include a ligation step to create self-circularized DNA fragments, which may be used to perform inverse PCR reactions. Such reactions allow known sequences to be used to amplify the unknown sequences to which known sequences are ligated (Zhao, Z., et al., Circular chromosome conformation capture (4C) uncovers extensive networks of epigenetically regulated intrachromosomal and interchromosomal interactions, Nature Genetics, 38(11) 1341-7 (2006)). Chromosome conformation capture carbon copy (“5C”) detects interactions between all restriction fragments within a given region typically where the region's size is no greater than one megabase (Dostie, J., et al., Chromosome Conformation Capture Carbon Copy (5C): a massively parallel solution for mapping interactions between genomic elements, Genome Research, 16(10) 1299-309 (2006)). High-throughput chromosome capture (“Hi-C”) uses high-throughput sequencing to identify the nucleotide sequence of fragments using paired end sequencing (Liberman-Aiden, E., et al., Comprehensive mapping of long-range interactions reveals folding principles of the human genome, Science, 326 (5950) 289-93 (2009)).

The present disclosure specifically modifies nucleic acid products generated by chromosomal conformation interaction techniques, such as those described herein, to spatially capture such nucleic acid products on a spatial array including a plurality of capture probes, where a capture probe of the plurality of capture probes includes a spatial barcode (e.g., as defined herein) and a capture domain.

### Methods of Detecting the Spatial Location of Chromosomal Conformation Interactions in a Biological Sample

Thus, provided herein are methods for determining the spatial location of chromosomal conformation interactions in a biological sample, the method including: (a) providing an array including a plurality of capture probes, where a capture probe of the plurality of capture probes includes: (i) a spatial barcode and (ii) a capture domain; (b) crosslinking accessible DNA in the biological sample; (c) fragmenting the crosslinked accessible DNA, thereby generating crosslinked fragmented DNA; (d) circularizing the crosslinked fragmented DNA, thereby generating crosslinked circularized DNA; (e) digesting the crosslinked circularized DNA, thereby generating crosslinked digested DNA; (f) incorporating a capture sequence onto the ends of the crosslinked digested DNA; (g) hybridizing the capture sequence of the crosslinked digested DNA to the capture domain of the capture probe on the array; and (h) determining (i) the sequence of the spatial barcode, or a complement thereof, and (ii) all or a portion of the sequence of the crosslinked digested DNA, or a complement thereof, and using the determined sequences of (i) and (ii) to determine the spatial location of chromosomal conformation interactions in the biological sample.

In some embodiments, the biological sample is disposed on the array (e.g., directly on the array). In some embodiments, the biological sample is disposed on a substrate. For example, the biological sample is disposed on a substrate that does not include a plurality of capture probes. In some embodiments, the method includes aligning the substrate including the biological sample with the array, such that at least a portion of the biological sample is aligned with at least a portion of the array (e.g., “sandwiching” as described herein).

In some embodiments, the accessible DNA is crosslinked in the biological sample with one or more crosslinking agents. Any suitable crosslinking agent can be used to crosslink the accessible DNA. Non-limiting examples of crosslinking agents include formaldehyde, paraformaldehyde, acetone, ethanol, methanol, or a combination thereof. Crosslinking the accessible DNA in the biological sample allows preservation of the chromosomal conformation interactions by physically locking in place portions of the genome that are interacting with one another, yet may be physically distant from each other in the linear genome.

After crosslinking the accessible DNA in the biological sample (e.g., a tissue section) the crosslinked accessible DNA may be fragmented. In some embodiments, fragmenting the crosslinked accessible DNA includes the use of a restriction enzyme, a DNase, and/or a micrococcal nuclease (MNase). Fragmenting the crosslinked accessible DNA breaks the DNA into smaller portions, which can be further processed as described herein.

In some embodiments, the DNase is one or more members of the DNase I family. In some embodiments, the DNase is one or more members of the DNase II family. In some embodiments, more than one DNase is used to fragment the crosslinked accessible DNA (e.g., a member of the DNase I family and a member of the DNase II family).

In some embodiments, a MNase enzyme is used to fragment the crosslinked accessible DNA. MNase is an enzyme derived from Staphylococcus aureus and is a relatively non-specific endo-exonuclease useful for the methods described herein.

Any suitable restriction enzyme can be used in the methods described herein. Cleavage methods and procedures for selecting restriction enzymes for cutting nucleic acid at specific sites are well known to the skilled artisan. For example, many suppliers of restriction enzymes provide information on conditions and types of DNA sequences cut by specific restriction enzymes, including New England Biolabs, Promega, Boehringer-Mannheim, and the like.

Restriction enzymes (i.e., restriction endonucleases) are traditionally classified into three types on the basis of subunit composition, cleavage position, sequence-specificity, and cofactor requirements. However, amino acid sequencing suggests extraordinary variety among restriction enzymes and revealed that, at the molecular level, there may be more than three different kinds.

Type I enzymes are complex, multi-subunit, combination restriction-and-modification enzymes that can cut DNA at random sites far from their recognition sequences. Type I enzymes do not produce discrete restriction fragments or distinct gel-banding patterns.

Type II enzymes can cut DNA at defined positions close to or within their recognition sequences. Type II enzymes can produce discrete restriction fragments and distinct gel banding patterns and are often used in various DNA analyses.

The most common Type II enzymes include HhaI, HindIII, and NotI that cleave DNA within their recognition sequences. Enzymes of this kind are available commercially. Most of these enzymes recognize DNA sequences that are symmetric because the enzymes bind to DNA as homodimers, but others (e.g., BbvCI: CCTCAGC) recognize asymmetric DNA sequences because these other enzymes bind as heterodimers. Some enzymes recognize continuous sequences (e.g., EcoRI: GAATTC) in which the two half-sites of the recognition sequence are adjacent, while others recognize discontinuous sequences in which the half-sites are separated (i.e., non-adjacent). Restriction enzyme cleavage results a 3′-hydroxyl on one side of each cut and a 5′-phosphate on the other. Restriction enzymes require only magnesium for activity and the corresponding modification enzymes require only S-adenosylmethionine. Modification enzymes tend to be small, with subunits in the 200-350 amino acid in length.

The next most common Type II enzymes, usually referred to as “Type IIS”, are those like FokI and AlwI that cleave outside of their recognition sequence to one side. These enzymes are intermediate in size, 400-650 amino acids in length, and they recognize sequences that are continuous and asymmetric. The enzymes have two distinct domains, one for DNA binding and the other for DNA cleavage. The domains may bind to DNA as monomers for the most part, but cleave DNA cooperatively by dimerization of the cleavage domains of adjacent enzyme molecules. For this reason, some type IIS enzymes may be more active on DNA molecules that contain multiple recognition sites. There is a wide variety of Type IIS restriction enzymes isolated from bacteria, phage, archaebacteria, and viruses of eukaryotic algae, which are commercially available (Promega, Madison, WI; New England Biolabs, Beverly, MA). Examples of Type IIS restriction enzymes that may be used with methods described herein include but are not limited to enzymes such as those listed in Table

TABLE 1
Examples of Type IIS restriction enzymes

| | Recognition |
|---|---|
| Enzyme-Source | (cleavage site) |
| Alw I-Acinetobacter lwoffii | GGATC(4/5) |
| Alw26 I-Acinetobacter lwoffi | GTCTC(1/5) |
| Bbs I-Bacillus laterosporus | GAAGAC(2/6) |
| Bbv I-Bacillus brevis | GCAGC(8/12) |
| BceA I-Bacillus cereus 1315 | IACGGC(12/14) |
| Bmr I-Bacillus megaterium | CTGGG(5/4) |
| Bsa I-Bacillus | GGTCTC(1/5) |
| stearothermophilus 6-55 | |
| Bst71 I-Bacillus | GCAGC(8/12) |
| stearothermophilus 71 | |
| BsmA I-Bacillus | GTCTC(1/5) |
| stearothermophilus A664 | |
| BsmB I-Bacillus | CGTCTC(1/5) |
| stearothermophilus B61 | |
| BsmF I-Bacillus | GGGAC(10/14) |
| stearothermophilus F | |
| BspM I-Bacillus species M | ACCTGC(4/8) |
| Ear I-Enterobacter aerogenes | CTCTTC(1/4) |
| Fau I-Flavobacterium aquatile | CCCGC(4/6) |
| Fok I-Flavobacterium | GGATG(9/13) |
| okeonokoites | |
| Hga I-Haemophilus gallinarum | GACGC(5/10) |
| Ple I-Pseudomonas lemoignei | GAGTC(4/5) |
| Sap I- | GCTCTTC(1/4) |
| Saccharopolyspora species | |
| SfaN I-Streptococcus | GCATC(5/9) |
| faecalis ND547 | |
| Sth 132 I-Streptococcus | CCCG(4/8) |
| thermophilus ST132^{a} | |

^{a}Gene, 195: 201-206 (1997).

A third major kind of Type II enzyme, more properly referred to as “Type IV,” are large, combination restriction-and-modification enzymes, 850-1250 amino acids in length, in which the two enzymatic activities reside in the same protein chain. These enzymes cleave outside of their recognition sequences; those that recognize continuous sequences (e.g., Eco57I: CTGAAG) cleave on just one side; those that recognize discontinuous sequences cleave on both sides and release a small fragment containing the recognition sequence. The amino acid sequences of these enzymes may be varied, but their organization is consistent. The enzymes comprise an N-terminal DNA-cleavage domain joined to a DNA-modification domain and one or two DNA sequence-specificity domains forming the C-terminus, or present as a separate subunit. When these enzymes bind to their substrates, the enzymes switch to either restriction mode to cleave the DNA, or modification mode to methylate the DNA.

As discussed above, the length of restriction recognition sites may vary. For example, the enzymes EcoRI, SacI, and SstI each recognizes a 6 base-pair (bp) sequence of DNA, whereas NotI recognizes a sequence 8 bp in length, and the recognition site for Sau3 AI is only 4 bp in length. Length of the recognition sequence may dictate how frequently the enzyme will cut within a random sequence of DNA. Enzymes with a 6 bp recognition site may cut at, on average, every 46 or 4096 bp; a 4 bp recognition site occurs roughly every 256 bp. The length of the restriction recognition site may also affect the resolution of the assay, i.e., shorter recognition sites may increase the resolution of the assay.

Different restriction enzymes can have the same recognition site—such enzymes are called isoschizomers. For example, the recognition sites for SacI and SstI are identical. In some cases, isoschizomers cut identically within their recognition site, but sometimes they do not. Isoschizomers may have different optimum reaction conditions, stabilities, and costs, which may influence the decision of which to use for methods described herein.

Restriction recognition sites can be unambiguous or ambiguous. The enzyme BamHI recognizes the sequence GGATCC and no others, and is therefore considered “unambiguous.” In contrast, HinfI recognizes a 5 bp sequence starting with GA, ending in TC, and having any base between. HinfI has an ambiguous recognition site. XhoII also has an ambiguous recognition site and will recognize and cut sequences of AGATCT, AGATCC, GGATCT, and GGATCC.

The recognition site for one enzyme may contain the restriction site for another. For example, a BamHI recognition site contains the restriction site for Sau3AI. Consequently, all BamHI sites can be cut using Sau3AI. Similarly, one of the four possible XhoII sites is also a recognition site for BamHI and all four XhoII sites can be cut using Sau3 AI.

Most recognition sequences are palindromes, i.e., the sequences read the same forward (5′ to 3′) and backward (3′ to 5′). Most, but certainly not all, recognition sites for commonly-used restriction enzymes are palindromes. Most restriction enzymes bind to their recognition site as dimers as described herein.

In some embodiments, the first restriction endonuclease cleavage site is 5′ (upstream) to the first spatial barcode and the first capture domain of the first capture probe. In some embodiments, the second restriction endonuclease cleavage site is 5′ (upstream) to the second spatial barcode and the second capture domain of the second capture probe.

In some embodiments, the array comprises a third set, a fourth set, or a fifth set of capture probes. In some embodiments, the array comprises about 1, about 2, about 3, about 4, about 5, about 6, about 7, about 8, about 9 sets, or more sets of capture probes. In some embodiments, each set of capture probes includes a unique release mechanism (e.g., restriction endonuclease cleavage site, photocleavable site, etc.).

In some embodiments, the sequence of the restriction enzyme recognition site is about 4 bp, about 5 bp, about 6 bp, about 7 bp, about 9 bp, about 9 bp, about 10 bp, about 11 bp, about 12 bp, about 13 bp, about 14 bp, about 15 bp, or more in length. In some embodiments, the sequence the restriction enzyme recognition site is about 4 bp to about 8 bp in length.

After the crosslinked accessible DNA is fragmented (e.g., fragmented by any of the methods described herein), the DNA fragment is circularized, thereby generating crosslinked circularized DNA. In some embodiments, circularizing the crosslinked fragmented DNA includes ligating together the two ends of the crosslinked fragmented DNA. In some embodiments, circularizing the crosslinked fragmented DNA includes a gap-fill reaction using one or more nucleotides (e.g., dNTPs: dATPs, dTTPs, dCTPs, dGTPs, dUTPs, etc.) followed by a ligation reaction. In some embodiments, at least one of the one or more nucleotides includes a biotin moiety. In some embodiments, the method includes an enrichment step where the biotin moiety interacts with a streptavidin moiety. In some embodiments, circularizing the crosslinked fragmented DNA (e.g., via ligation or a gap-fill reaction followed by ligation) includes the use of a ligase.

In some embodiments, the ligase is one or more of a T4 RNA ligase (Rnl2), CircLigase, a PBCV-1 DNA ligase, a Chlorella virus DNA ligase, a single-stranded DNA ligase, a T4 DNA ligase, and a combination thereof.

The crosslinked circularized DNA may then be digested, thereby generating crosslinked digested DNA. In some embodiments, the digesting includes the use of one or more restriction enzymes (e.g., any of the restriction enzymes described herein). In some embodiments, the digesting includes the use of one or more DNases (e.g., any of the DNases described herein). In some embodiments, the digesting includes the use of one or more restriction enzymes and one or more DNases. Optionally, before, during, or after digesting the crosslinked circularized DNA, the method may include treating the biological sample with an exonuclease. In some embodiments, the exonuclease is Exonuclease VIII. Exonuclease VIII can be used to preferentially degrade single-stranded nucleic acids. Thus, single-stranded nucleic acids present in the biological sample can be degraded leaving double-stranded crosslinked digested DNA behind. Removal single-stranded nucleic acids can improve the specificity of the assay by reducing the likelihood of undesirable interactions of single-stranded nucleic acids in the sample in further steps of the assay (e.g., incorporating a capture sequence).

In some embodiments, the crosslinked digested DNA is decrosslinked prior to adding to the capture sequence as described below.

A capture sequence can be incorporated (e.g., added) onto an end (e.g., both ends) of the crosslinked digested DNA. Various methods may be used to add a capture sequence (e.g., a sequence capable of hybridizing to a capture domain of a capture probe on a spatial array) to nucleic acids (i.e., crosslinked digested DNA). In some embodiments, incorporating the capture sequence onto the ends of the crosslinked digested DNA includes ligating a poly(A) oligonucleotide onto the ends of the crosslinked digested DNA. For example, a plurality of poly(A) oligonucleotides and a ligase (e.g., any of the ligases provided herein) can be contacted with the biological sample. The ligase enzyme may ligate a poly(A) oligonucleotide onto the ends of the crosslinked digested DNA. The poly(A) oligonucleotide can be about 5, about 6, about 7, about 8, about 9, about 10, about 11, about 12, about 13, about 14, about 15, about 16, about 17, about 18, about 19, about 20, about 21, about 22, about 23, about 24, about 25 nucleotides, or more nucleotides in length. The poly(A) oligonucleotide may only need to be sufficiently long (e.g., about 5 nucleotides) to hybridize the capture domain (e.g., a poly(T) sequence) of a capture probe.

Alternatively, a capture sequence can be added onto the ends of the crosslinked digested DNA using a terminal transferase and a plurality of dATPs. In some embodiments, the terminal transferase is terminal deoxynucleotidyl transferase. Terminal transferases may be used to add nucleotides (e.g., dATPs) in a template-independent manner. In some embodiments, a terminal transferase adds one or more dATPs (e.g., about 5, about 6, about 7, about 8, about 9, about 10, about 11, about 12, about 13, about 14, about 15, about 16, about 17, about 18, about 19, about 20, about 21, about 22, about 23, about 24, or about 25 dATPs) onto the ends of the crosslinked digested DNA, thereby generating a poly(A) sequence. The generated poly(A) sequence only needs to be sufficiently long (e.g., about 5 nucleotides) to hybridize the capture domain (e.g., a poly(T) sequence) of a capture probe.

In some embodiments, the capture probe includes one or more functional domains, a unique molecular identifier (as defined herein), a cleavage domain, or a combination thereof. In some embodiments, the one or more functional domains includes a primer binding site or a sequencing specific site.

In some embodiments, the capture sequence can be a randomer sequence, for example. by ligating a randomer capture sequence onto the crosslinked digested DNA using a ligase (e.g., any of the ligases described herein). Alternatively, terminal deoxynucleotidyl transferase and dNTPs can be used to randomly add nucleotides to the crosslinked digested DNA. The capture domain on a capture probe may also be a random sequence, which can hybridize to the randomers appended to the ends of the crosslinked digested DNA for hybridization and capture.

In some embodiments, the capture sequence can be a fixed or known sequence. For example, a capture sequence could be an oligonucleotide that is a non-random combination of nucleotides which is ligated on the ends of crosslinked digested DNA. The capture domain of a capture probe on an array, or a subset thereof, can be a complement to the non-random oligonucleotide appended to the ends of the crosslinked digested DNA for hybridization and capture.

In some embodiments, the capture domain of a capture probe on the array includes a poly(T) sequence.

In some embodiments, the array includes a plurality of features selected from the group consisting of: a spot, an inkjet spot, a masked spot, a pit, a post, a well, a ridge, a divot, a hydrogel pad, and a bead.

In some embodiments, the sequence of the spatial barcode is unique to a distinct position on the array. For example, each feature includes capture probes comprising a spatial barcode that is the same for that feature, and each feature therefore includes a unique spatial barcode.

In some embodiments, the determining includes sequencing. In some embodiments, the sequencing includes high-throughput sequencing. In some embodiments, the determining includes fluorescence detection.

In some embodiments, the biological sample is a tissue sample. In some embodiments, the tissue sample is a fixed tissue sample. In some embodiments, the tissue sample is a fresh-frozen tissue sample. In some embodiments, the tissue sample is a tissue section. In some embodiments, the tissue section is a fresh-frozen tissue section. In some embodiments, the tissue section is a fixed tissue section.

In some embodiments, the biological sample is a diseased biological sample. In some embodiments, the biological sample is a healthy biological sample.

In some embodiments, the fixed tissue section is a formalin-fixed paraffin-embedded (FFPE) tissue section, a paraformaldehyde-fixed tissue section, an acetone-fixed tissue section, a methanol-fixed tissue section, or an ethanol-fixed tissue section. In some embodiments, the FFPE tissue section is deparaffinized and decrosslinked prior to (b).

In some embodiments, the method includes staining the biological sample. In some embodiments, the staining includes hematoxylin and/or eosin staining. In some embodiments, the staining includes use of a radioisotope, a fluorophore, a chemiluminescent compound, a bioluminescent compound, or a combination thereof.

In some embodiments, the method includes imaging the biological sample.

In some embodiments, the method includes permeabilizing the biological sample. In some embodiments, the permeabilizing includes use of a protease, a surfactant, and/or a detergent. In some embodiments, the protease includes Proteinase K, pepsin, and/or collagenase.

### Kits

The present disclosure also features kits for performing any of the methods described herein. Such kits include at least a spatial array including a plurality of capture probes, means for digesting DNA (e.g., restriction enzymes, DNases, micrococcal nucleases, etc.), and a ligase. Kits of the present disclosure can also include instructions for performing any of the methods described herein.

Thus, provided herein are kits including: (a) a spatial array including a plurality of capture probes, where a capture probe of the plurality of capture probes includes: (i) a spatial barcode (e.g., a spatial barcode as defined herein) and (ii) a capture domain (e.g., any of the capture domains described herein); (b) one or more restriction enzymes, a DNase, and/or an MNase; (c) a ligase; and (d) Exonuclease VIII.

In some embodiments, the kit includes the one or more restriction enzymes. In some embodiments, the one or more restriction enzyme includes AlwI, Alw26, BamHI, BbsI, BbvI, BceAI, BmrI, Bsal, Bst71, BsmAI, BsmBI, BsmFI, BspMI, EarI, FauI, FokI, HgaI, PleI, SapI, SfaNI, or Sth132. In some embodiments, the DNase includes one or both of a DNase I enzyme and a DNase II enzyme.

In some embodiments, the kit includes the DNase. In some embodiments, the kit includes the MNase. In some embodiments, the kit includes a polymerase.

In some embodiments, the kit includes one or more nucleotides. In some embodiments, at least one of the one or more nucleotides includes a biotin moiety. In some embodiments, the biotin moiety can interact with a streptavidin moiety (e.g., a streptavidin molecule immobilized on a bead, a plate, etc.).

In some embodiments, the kit includes a plurality of poly(A) oligonucleotides. In some embodiments, the kit includes a terminal transferase enzyme. Any suitable transferase enzyme can be used, including, for example, terminal deoxynucleotidyl transferase. In some embodiments, the kit includes a plurality of dATPs.

In some embodiments, the kit includes one or more crosslinking agents. In some embodiments, the one or more crosslinking agents includes formaldehyde, PFA, formalin, methanol, or acetone.

### Compositions

The present disclosure also features compositions associated with any of the methods described herein. Such compositions include those described herein and/or exemplified by the drawings described herein.

Thus, provided herein are compositions including: a) a spatial array including a plurality of capture probes, where a capture probe of the plurality of capture probes includes: (i) a spatial barcode and (ii) a capture domain; b) a crosslinking agent; and c) crosslinked accessible DNA.

Also provided herein are compositions including: a) a spatial array including a plurality of capture probes, where a capture probe of the plurality of capture probes includes: (i) a spatial barcode and (ii) a capture domain; b) one or more restriction enzymes (e.g., any of the restrictions enzymes described herein), a DNase (e.g., any of the DNases described herein), and a single-stranded exo-endonuclease, such as MNase; and c) crosslinked fragmented DNA.

Also provided herein are compositions including: a) a spatial array including a plurality of capture probes, where a capture probe of the plurality of capture probes includes: (i) a spatial barcode and (ii) a capture domain; b) one or more restriction enzymes and/or a DNase; and c) crosslinked circularized DNA.

In some embodiments, the one or more restriction enzymes is selected from the group including: AlwI, Alw26, BamHI, BbsI, BbvI, BceAI, BmrI, Bsal, Bst71, BsmAI, BsmBI, BsmFI, BspMI, EarI, FauI, FokI, HgaI, PleI, SapI, SfaNI, or Sth132.

In some embodiments, the composition includes a ligase. In some embodiments, the ligase is one or more of a T4 RNA ligase (Rnl2), a PBCV-1 DNA ligase, a Chlorella virus DNA ligase, a single-stranded DNA ligase, a T4 DNA ligase, and combinations thereof. In some embodiments, the composition includes a polymerase (e.g., a DNA polymerase).

In some embodiments, the composition includes the DNase. In some embodiments, the composition includes the MNase.

In some embodiments, the composition includes one or more dNTPs (e.g., dATPs, dTTPs, dCTPs, dGTPs, dUTPs, etc.). In some embodiments, at least one dNTP of the one or more dNTPs includes a biotin moiety. In some embodiments, the biotin moiety of the one or more dNTPs specifically binds a streptavidin moiety. In some embodiments, the streptavidin moiety is immobilized on a plate, a bead, etc.

In some embodiments, the composition includes an exonuclease. In some embodiments, the exonuclease preferentially degrades single-stranded nucleic acids. In some embodiments, the exonuclease is Exonuclease VIII.

Also provided herein are compositions including: a) a spatial array including a plurality of capture probes, where a capture probe of the plurality of capture probes includes: (i) a spatial barcode and (ii) a capture domain; b) crosslinked digested DNA; and c) either: (i) a ligase and a plurality of poly(A) oligonucleotides, or (ii) a terminal transferase and a plurality of dATPs.

In some embodiments, the poly(A) oligonucleotide is a capture sequence capable of hybridizing to a capture domain of a capture probe on a spatial array. In some embodiments, the poly(A) oligonucleotide is ligated (e.g., ligated with any of the ligases described herein) to the crosslinked digested DNA. In some embodiments, the terminal transferase (e.g., terminal deoxynucleotidyl transferase) incorporates (e.g., adds) a plurality of dATP molecules to the end of the crosslinked digested DNA, thereby by generating a poly(A) sequence (e.g., a capture sequence) that can hybridize to a capture domain of a capture probe on a spatial array.

Also provided herein are compositions including: a) a spatial array including a plurality of capture probes, where a capture probe of the plurality of capture probes includes: (i) a spatial barcode and (ii) a capture domain; and b) crosslinked digested DNA hybridized to the capture domain of the capture probe via a capture sequence.

Also provided herein are compositions including: a) a spatial array including a plurality of capture probes, where a capture probe of the plurality of capture probes includes: (i) a spatial barcode and (ii) a capture domain; (b) one or more restriction enzymes, a DNase, and/or a MNase; (c) a ligase; and (d) Exonuclease VII.

In some embodiments, the composition includes the one or more enzymes. In some embodiments, the one or more restriction enzymes include AlwI, Alw26, BamHI, BbsI, BbvI, BceAI, BmrI, Bsal, Bst71, BsmAI, BsmBI, BsmFI, BspMI, EarI, FauI, FokI, HgaI, PleI, SapI, SfaNI, or Sth132.

In some embodiments, the composition includes the DNase. In some embodiments, the composition includes the MNase. In some embodiments, the composition includes a polymerase.

In some embodiments, the composition includes one or more nucleotides. In some embodiments, the one or more nucleotides includes a biotin moiety. In some embodiments, the one or more nucleotides are one or more dNTPs.

In some embodiments, the composition includes a plurality of poly(A) oligonucleotides.

In some embodiments, the composition includes a terminal transferase enzyme. In some embodiments, the composition includes a plurality of dATPs.

In some embodiments, the composition includes one or more crosslinking agents. In some embodiments, the one or more crosslinking agents includes formaldehyde.

### Library Preparation

In some embodiments, the crosslinked digested DNA (e.g., captured crosslinked digested DNA), and/or amplicons of such products, can be prepared for downstream applications, such as generation of a sequencing library and next-generation sequencing. Generating sequencing libraries are known in the art. For example, the crosslinked digested DNA can be purified and collected for downstream amplification steps. The amplification products can be amplified using PCR, where primer binding sites flank the spatial barcode and target nucleic acid, or a complement thereof, generating a library associated with a particular spatial barcode. In some embodiments, the library preparation can be quantitated and/or quality controlled to verify the success of the library preparation steps. The library amplicons are sequenced and analyzed to decode spatial information of the crosslinked digested DNA.

Alternatively, or additionally, the amplicons can then be enzymatically fragmented and/or size-selected in order to provide for desired amplicon size. In some embodiments, when utilizing an Illumina® library preparation methodology, for example, P5 and P7, sequences can be added to the amplicons thereby allowing for capture of the library preparation on a sequencing flow cell (e.g., on Illumina sequencing instruments). Additionally, i7 and i5 can index sequences be added as sample indexes if multiple libraries are to be pooled and sequenced together. Further, Read 1 and Read 2 sequences can be added to the library for sequencing purposes, if not already present on the capture probe on the array, and incorporated into the captured crosslinked and digested DNA. The aforementioned sequences can be added to a library preparation sample, for example, via End Repair, A-tailing, Adaptor Ligation, and/or PCR. The cDNA fragments can then be sequenced using, for example, paired-end sequencing using TruSeq Read 1 and TruSeq Read 2 as sequencing primer sites, although other methods are known in the art and may be used with the methods described herein.

### EXAMPLES

### Example 1. Methods of Spatially Detecting Chromosomal Conformation Interactions in a Biological Sample

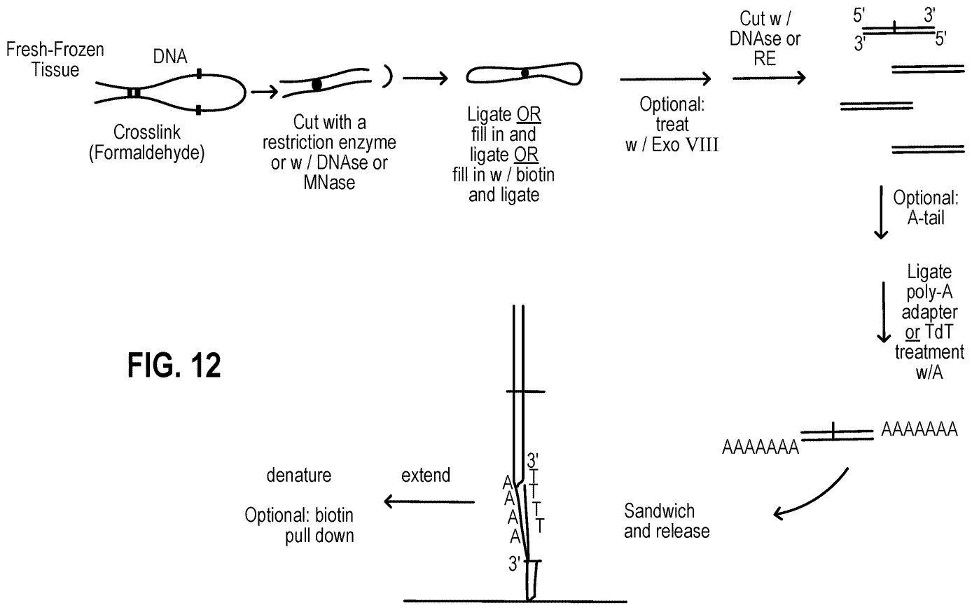

The present disclosure features exemplary methods of spatially detecting chromosomal conformation interactions in a biological sample (e.g., a tissue section). FIG. 12 shows an exemplary workflow for detecting spatial chromosomal conformation interactions. As shown from left to right, DNA (e.g., genomic DNA, accessible DNA, etc.) in a biological sample (e.g., a tissue section) is crosslinked with one or more crosslinking agents (e.g., formaldehyde, paraformaldehyde, etc.).

Next, the crosslinked accessible DNA is fragmented, thereby generating crosslinked fragmented DNA. The fragmenting can be accomplished via various methods. For example, fragmenting can be performed with one or more of a restriction enzyme (e.g., one or more of any of the restriction enzymes described herein), a DNase (e.g., any of the DNases described herein), a micrococcal nuclease (MNase), and a combination thereof.

The resulting crosslinked fragmented DNA is circularized, thereby generating crosslinked circularized DNA. Non-limiting examples of circularizing the crosslinked fragmented DNA includes ligating (e.g., ligating with any of the ligases described herein) the two ends of the crosslinked fragmented DNA or performing a gap-fill reaction with one or more nucleotides followed by ligation. In some examples, where a gap-fill reaction is performed, at least one of the one or more nucleotides includes a biotin moiety. The biotin moiety is capable of interacting with an immobilized streptavidin molecule. For example, the streptavidin moiety can be immobilized on a feature, such as a bead, a well, a plate, etc. The interaction between the biotin moiety of incorporated nucleotide (e.g., incorporated during the gap-fill reaction) and the streptavidin moiety provides as an optional enrichment step to selectively isolate the crosslinked circularized DNA (e.g., accessible DNA, genomic DNA, etc.). In some examples, after generating the crosslinked circularized DNA, optionally, the biological sample can be treated with an exonuclease (e.g., Exonuclease VIII). Exonuclease VIII can preferentially degrade single-stranded nucleic acids in the biological sample, thus leaving behind the crosslinked circularized DNA for further processing and improving the specificity of the assay.

Next, the crosslinked circularized DNA is digested to thereby generate crosslinked digested DNA. Non-limiting examples of digestion methods include digestion using one or more restriction enzymes (e.g., any of the restriction enzymes described herein) and/or one or more DNases (e.g., any of the DNases described herein). The resulting crosslinked digested DNA in the biological sample (e.g., a tissue section) can be captured by a capture probe on a spatial array. In some examples, the crosslinked digested DNA can be captured by a capture domain including a random capture domain (e.g., random nucleotides such as a random hexamer, a random decamer, etc.). The random sequence of the capture domain can interact with (e.g., hybridize to) the free ends of the crosslinked digested DNA and capture the crosslinked digested DNA on the array.

In other examples, a capture sequence is incorporated onto the two ends of the crosslinked digested DNA. The capture sequence can be any sequence so long as the capture sequence is complementary to the capture domain of the capture probe on the array. In some examples, the capture sequence is incorporated (e.g., added) onto the ends of the crosslinked digested DNA via ligation (e.g., with any of the ligases described herein). For example, a poly(A) oligonucleotide is ligated onto the ends of the crosslinked digested DNA. The poly(A) oligonucleotide may function as a capture sequence that can hybridize to the capture domain of a capture probe on the array. In such examples, the capture domain comprises a poly(T) sequence. Alternatively, the capture sequence may be incorporated (e.g., ligated) onto the ends of the crosslinked digested DNA using a transferase enzyme and a plurality of dATPs. The transferase enzyme (e.g., a terminal deoxynucleotidyl transferase) can append dATP molecules onto the ends of the crosslinked digested DNA. The appended dATP molecules function as a capture sequence that is complementary to the capture domain of the capture probe on the array. In such examples, the capture domain comprises a poly(T) sequence.

In another example, a non-random capture sequence comprising an oligonucleotide of a known series of nucleotides (dNTPs: As, Ts, Cs, and Gs) can also be incorporated on the ends of the crosslinked digested DNA by ligation of the non-random oligonucleotide to the ends of the crosslinked digested DNA. In such examples, the capture probe on the array comprises a capture domain which is complementary to the oligonucleotide appended to the ends of the crosslinked digested DNA.

The captured crosslinked digested DNA can undergo additional reactions to determine the sequence of the spatial barcode, or a complement thereof, of the capture probe and all or a portion of the sequence of the crosslinked digested DNA, or a complement thereof. For example, the capture probe can be extended using the crosslinked digested DNA as a template, thereby generating an extended capture probe. In some examples, the crosslinked digested DNA is extended using the capture probe as a template (e.g., extended towards the 5′ end of the capture probe), thereby incorporating a complement of the spatial barcode into the crosslinked digested DNA. In some embodiments, the extended crosslinked digested DNA is denatured from the extended capture probe (e.g., denatured via heat, KOH, etc.). The denatured products can optionally be further amplified and prepared as a sequencing library as described herein.

What is claimed is:

1. A method for analyzing chromosomal conformation interactions in a biological sample, the method comprising:
(a) providing an array comprising a plurality of capture probes, wherein a capture probe of the plurality of capture probes comprises: (i) a spatial barcode and (ii) a capture domain;
(b) crosslinking accessible DNA in the biological sample;
(c) fragmenting the crosslinked accessible DNA, thereby generating crosslinked fragmented DNA;
(d) circularizing the crosslinked fragmented DNA, thereby generating crosslinked circularized DNA;
(e) digesting the crosslinked circularized DNA, thereby generating crosslinked digested DNA;
(f) incorporating a capture sequence onto an end of the crosslinked digested DNA; and
(g) hybridizing the capture sequence of the crosslinked digested DNA to the capture domain of the capture probe on the array.

2. The method of claim 1, wherein the biological sample is disposed on the array.

3. The method of claim 1, wherein the biological sample is disposed on a substrate, wherein the method further comprises aligning the substrate comprising the biological sample with the array, such that at least a portion of the biological sample is aligned with at least a portion of the array.

4. The method of claim 1, wherein the digesting the crosslinked circularized DNA comprises treating the biological sample with Exonuclease VIII.

5. The method of claim 1, wherein the circularizing the crosslinked fragmented DNA comprises ligating together ends of the crosslinked fragmented DNA.

6. The method of claim 1, wherein the circularizing the crosslinked fragmented DNA comprises a gap-fill reaction with one or more nucleotides.

7. The method of claim 6, wherein at least one of the one or more nucleotides comprises a biotin moiety, optionally, further comprising an enrichment step wherein the biotin moiety interacts with a streptavidin moiety.

8. The method of claim 1, wherein the incorporating the capture sequence onto the end of the crosslinked digested DNA comprises ligating a poly(A) oligonucleotide onto the end of the crosslinked digested DNA.

9. The method of claim 1, wherein the crosslinking the accessible DNA in the biological sample comprises use of formaldehyde.

10. The method of claim 1, wherein the fragmenting the crosslinked accessible DNA in (c) or the digesting in (e) comprises use of a restriction enzyme, a DNase, and/or a MNase.

11. The method of claim 10, wherein the restriction enzyme is AlwI, Alw26, BamHI, BbsI, BbvI, BceAI, BmrI, Bsal, Bst71, BsmAI, BsmBI, BsmFI, BspMI, EarI, FauI, FokI, HgaI, PleI, SapI, SfaNI, or Sth132.

12. The method of claim 1, wherein the capture probe further comprises one or more functional domains, a unique molecular identifier, a cleavage domain, or a combination thereof.

13. The method of claim 1, wherein the capture domain comprises a poly(T) sequence.

14. The method of claim 1, wherein the method further comprises (h) determining (i) the sequence of the spatial barcode, or a complement thereof, and (ii) all or a portion of the sequence of the crosslinked digested DNA, or a complement thereof, and using the determined sequences of (i) and (ii) to determine the spatial location of chromosomal conformation interactions in the biological sample.

15. The method of claim 14, wherein the determining in (h) comprises sequencing.

16. The method of claim 14, wherein the determining in (h) comprises fluorescent detection.

17. The method of claim 1, wherein the biological sample is a tissue sample.

18. The method of claim 17, wherein the tissue sample is a tissue section.

19. The method of claim 1, wherein the method further comprises staining the biological sample and/or imaging the biological sample.

20. The method of claim 1, wherein the method further comprises permeabilizing the biological sample.
